(19) United States
(12) Patent Application Publication
(10) Pub. No.: US 2024/0238562 A1
(43) Pub. Date: Jul. 18, 2024

(54) Catheter

(71) Applicant: Jason Oehler, Elmhurst, IL (US)

(72) Inventor: Jason Oehler, Elmhurst, IL (US)

(21) Appl. No.: 18/412,224

(22) Filed: Jan. 12, 2024

Related U.S. Application Data
(60) Provisional application No. 63/479,588, filed on Jan. 12, 2023.

(51) Int. Cl. A61M 25/02

(52) U.S. Cl. CPC A61M 25/02; A61M 2025/0286; A61M 2205/0216

(57) ABSTRACT

The disclosed invention relates to an improved catheter designed to enhance functionality and patient comfort during medical procedures. The catheter comprises a main tube with detachable casing portions, facilitating modular adjustments, and features at least one first canal and at least one second canal for fluid management. Notably, an arcuate feature in the at least one static duct allows the at least one static tube to be essentially perpendicular to the main tube, preventing external hoses from causing discomfort to the patient. The resilient casing, a fastener for detachable connections, and versatile fluid conduits make this catheter a user-friendly and adaptable tool for medical practitioners across various applications.

### CROSS-REFERENCE TO RELATED APPLICATIONS

The present application claims the benefit of U.S. Provisional Patent Application No. 63/479,588, filed Jan. 12, 2023, the entirety of which is hereby incorporated by reference.

### BACKGROUND OF THE INVENTION

### Field of the invention

One major problem with existing catheter hubs, or casings, is that they are configured in a straight line with the catheter's main tube, generally aligned with the catheter's longitudinal axis. When this design is used in the neck veins, for example, it is problematic for the patient while the catheter is indwelling. This axial alignment typically requires the external portions used with the catheter, all the hoses and clips involved with inserting wires and injecting medicines into a patient, to be situated high on the patient's neck. This is uncomfortable as the dressings of the catheter also sit in the mid or upper neck area. Unfortunately, while the catheter is essential to the care the patient is receiving, the existing catheters also add greatly to the discomfort of the patient.

There is a need in the industry for an apparatus, such as a catheter, that has a special hub or casing that can eliminate the hose, clips, needles, wires, and other catheter accessories from lying near a patient's face. I noticed that this type of catheter with a casing that provides comfort to patients is currently unavailable in the industry.

### SUMMARY

The present invention is directed to an apparatus that satisfies this need and relates to the external components of commonly placed catheters including multiple port venous access catheters. Existing catheter hubs, casings of resilient material usually, typically attach to the exterior portion of a catheter. The function of this hub is to allow an external component of a catheter to be attached to fluid delivery or aspiration device such as syringe or tubing. This component also functions as a part of the insertion process which allows for guide wire access to a patient's vein. This is also important in the situation where a catheter is to be exchanged over the wire, as is common in the medical industry. I envisioned a catheter that maintains full functionality while being more comfortable for the patient.

The hub, casing, is designed to allow for locating the external portions of a catheter at its hub away from, best mode is approximately ninety degrees, the longitudinal axis of the catheter's main tube. One key component is a moveable portion of the casing that encases ducts, connected to tubes, and is partially separable from a static portion of the casing with other ducts connected to other tubes. The inner lumen, the ducts, of the moveable portion remains patent, unblocked, while being sufficiently aligned with the catheter's longitudinal axis. This is important for functions such as catheter insertion over the wire or catheter exchange over a wire.

This partially separable portion, the moveable portion of the casing with the ducts and tubes, can be manufactured with a partial perforation seam or a partial thinning of the hub material that can be torn or pulled away from the main body to a point that it cannot be separated further. Depending upon the material used, this port could return to its original position when the over the wire procedure is completed. The lumen of this port, in this case the ducts and the tubes, will be sized to accept a standard sized guide wire while situated in the straight or curved orientation.

With that in mind, the present invention discloses an improved catheter designed to enhance functionality and adaptability in medical procedures. The improved catheter comprises a main tube with a first main tube end integrally attached to a casing. The main tube incorporates at least one first canal and at least one second canal, offering versatility in fluid management.

The casing is a pivotal component, consisting of a moveable portion with a first sidewall and a static portion with a second sidewall. These portions are designed for detachable connection, providing flexibility and ease of assembly. Enclosed within the moveable portion is at least one moveable duct, while the static portion houses at least one static duct. Additionally, the catheter features at least one static tube integrally attached to the static duct and at least one moveable tube integrally attached to the moveable duct. The alignment of the moveable tube, moveable duct, and second canal forms a conduit, as does the alignment of the static tube, static duct, and first canal.

Key features include the detachable connection between the first sidewall and the second sidewall, allowing for modular adjustments. Furthermore, the at least one static tube is positioned with a longitudinal axis perpendicular to the main tube's longitudinal axis, enhancing the catheter's adaptability for specific medical applications. It is understood that, though the tubes used with a catheter are flexible, they generally have a longitudinal axis when straightened. Too, though the ducts are arcuate, their best mode is to turn generally by a ninety-degree angle from the main tube. I expect that any angle could be used so long as it provides the same solution to the problem.

The casing, crucial to the catheter's structure, is constructed from a resilient material, ensuring durability and flexibility. This resilience allows the at least one moveable portion to spring back to its original position, i.e., alongside the at least one static portion. To facilitate secure attachment, an optional fastener can be incorporated, connecting the first sidewall and the second sidewall.

Crucially, the invention incorporates an arcuate feature in the at least one static duct, allowing the at least one static tube to be sufficiently perpendicular to the main tube thus allowing for placing external pieces out of the way of the patient area. Likewise for the at least one moveable tube and the at least one moveable duct. This design innovation is particularly advantageous as it prevents external hoses from inconveniencing the patient, eliminating any potential discomfort or interference during medical procedures that use a catheter.

In another embodiment, my improved catheter is made up of the catheter having a main tube, a casing, and a first main tube end. The first main tube end being integrally attached to the casing, I envision this being done with a manufacturing process that is common in the plastic industry, or the like.

This main tube has at least one first canal and at least one second canal. In my experience catheters have one or many canals depending on the procedure being performed. The casing is made having a moveable portion and a static portion and it is made from a resilient material, plastic, soft metal, rubber, fiberglass, paper fibers to name a few examples. Its moveable portion has a first sidewall and the static portion has a second sidewall, the first sidewall and the second sidewall can be connected in such a way as to be detachable. I envision that connection of sidewalls occurring, the best way, from the properties of the materials the casing is made. So, the material is resilient and though the casing might be formed with the portions adjacent to one another, the resiliency of the material allows the portions to be pulled apart from each other. Other methods of attachment are possible.

This embodiment has at least one moveable duct and at least one static duct. The at least one moveable duct being enclosed in the moveable portion of the casing. Too, the at least one static duct is enclosed in the static portion. Certainly, there may be some outside of the enclosure but best if fully enclosed. Being integrally attached to the static duct is at least one static tube that has a longitudinal axis sufficiently perpendicular to the main tube's longitudinal axis, turning away from the main tube, something not found in today's catheters. Likewise, at least one moveable tube is integrally attached to the at least one moveable duct.

The invention is made such that the at least one moveable tube and the at least one moveable duct and the at least one second canal are aligned to form a conduit. The at least one static tube and the at least one static duct and the at least one first canal being aligned to form another conduit. These conduits are how medicine flows through the catheter.

To facilitate use, there is at least one suture ring integrally attached to the casing. Also, there may be a fastener incident to the first sidewall and the second sidewall, allowing for a sort of tearing apart of the portions at a junction, or seam.

In conclusion, the improved catheter presents a novel design with detachable casing portions, versatile fluid conduits, and a resilient structure, providing medical practitioners with an advanced tool for various medical procedures that require the use of a catheter. The unique features of the catheter enhance adaptability, ease of use, and overall efficiency in medical applications. It also introduces an arcuate feature for optimal patient comfort, making it an advanced and patient-friendly tool for medical practitioners.

### BRIEF DESCRIPTION OF THE SEVERAL VIEWS OF THE DRAWINGS

These and other features, aspects and advantages of the present invention will become better understood with regard to the following description, appended claims, and accompanying drawings where:

FIG. 1 shows a perspective view of an embodiment of the present invention.

FIG. 2 shows a perspective view of another embodiment of the present invention being used on a patient.

### DESCRIPTION

### Overview.

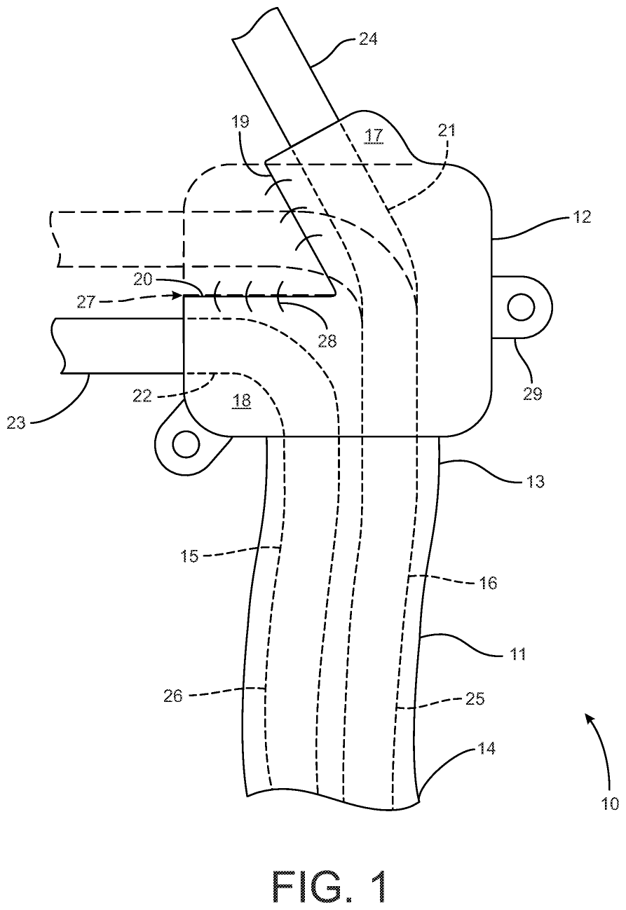

As shown in FIG. 1, a perspective view of an embodiment of the present invention comprises an apparatus that is an improved catheter 10. It has a main tube 11 that is typical of catheters used in the healthcare industry. There is shown a casing 12 that I envision being made of a resilient material, plastic or rubber or something similar. Today's catheters use such a material for the manufacture of their hubs.

The main tube has a first main tube end 13 that is integrally attached to the casing 12 and could be made of a different material if needed. This main tube has at least one first canal 15 and it also has at least one second canal 16. These canals are commonly found in today's catheters and facilitate, via a long conduit, the delivery of medicines and wires, to name a few examples, to the patient end of the catheter 14.

Shown is the casing having a moveable portion 17 and a static portion 18. The moveable portion 17 is shown to have a first sidewall 19 and the static portion has a second sidewall 20. Shown enclosed in the moveable portion 17 is at least one moveable duct 21. There is also shown at least one static duct 22 that is enclosed in the static portion 18.

Also shown is an at least one static tube 23 being integrally attached to the at least on static duct 22. And an at least one moveable tube 24 being integrally attached to the at least one moveable duct 21.

As shown in FIG. 1, conduits are formed. One by the at least one moveable tube 24 and the at least one moveable duct 21 and the at least one second canal 16 all three being aligned to form a conduit 25. Another shown is by the at least one static tube 23 and the at least one static duct 22 and the at least one first canal 15 also being aligned to form another conduit 26. These patent conduits are how medicines or wires, or other appropriate items are passed via the catheter into a patient's body.

The figure shows an embodiment where the first sidewall 19 and the second sidewall 20 are detachably connected. This can be accomplished by material properties, a snugly fitting form of a resilient material for instance, where the sidewalls 19, 20 are held in an adjacent position (shown by the alternate position) forming a seam or junction 27 but the moveable portion 17 can be torn or pulled away from the static portion 18. Once no longer being pulled or held away, the moveable portion 17 would spring back to the adjacent position.

Also shown in FIG. 1 is the at least one static tube 23 having a longitudinal axis generally and sufficiently perpendicular to the main tube's 11 longitudinal axis. Too there is shown an optional fastener 28 that is incident to the first sidewall 19 and the second sidewall 20. A healthcare provider could attach the improved catheter 10 to a patient's skin or gown (not shown) via the suture rings 29, stitched or with some sort of adhesive, attached to the casing 12.

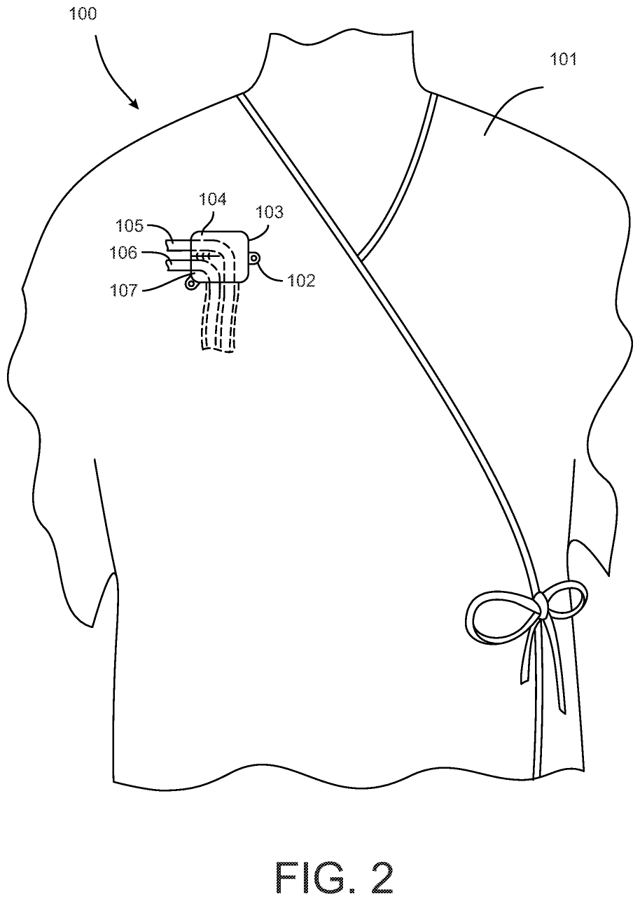

As shown in FIG. 2, a perspective view of another embodiment of the present invention being used on a patient comprises an improved catheter 100. It is attached to a patient's gown 101, could also be to the skin, via suture rings 102. Notably shown is the casing 103 having its moveable portion 104 and its static portion 107 with their respective sidewall (FIGS. 1, 19 and 20) in an adjacent position. This configuration depicts how the arcuate nature of the at least one moveable duct 105 and the at least one static duct 106 together with their respective tubing (FIGS. 1, 24 and 23) allow the catheter accessories to be out of the patient's face and head or other patient area, the groin for instance or other undesirable parts of the body that might increase the chances for infections.

Although the present invention has been described in considerable detail with the reference to certain preferred versions thereof, other versions are possible. For instance, the catheter may incorporate a single canal with multiple ducts and tubes, the tubes may be partially enclosed in the casing, or hinges and springs could be used instead of a resilient material to cause the moveable portion to spring back into place. Therefore, the spirit and scope of the appended claims should not be limited to the description of the preferred versions contained herein.

Any element in a claim that does not explicitly state “means for” performing a specified function, or “step for” performing a specific function, is not to be interpreted as a “means” or “step” clause as specified in 35 U.S.C. § 112, ¶ 6. In particular, the use of “step of” in the claims herein is not intended to invoke the provisions of 35 U.S.C. § 112, ¶ 6.

What I claim is:

1. An improved catheter comprising:
the catheter having a main tube;
the catheter having a casing;
the main tube having a first main tube end;
the first main tube end being integrally attached to the casing;
the main tube having at least one first canal;
the main tube having at least one second canal;
the casing having a moveable portion;
the casing having a static portion;
the moveable portion having a first sidewall;
the static portion having a second sidewall;
at least one moveable duct;
at least one static duct;
the at least one moveable duct being enclosed in the moveable portion;
the at least one static duct being enclosed in the static portion;
at least one static tube;
at least one moveable tube;
the at least one static tube being integrally attached to the at least on static duct;
the at least one moveable tube being integrally attached to the at least one moveable duct;
the at least one moveable tube and the at least one moveable duct and the at least one second canal being aligned to form a conduit;
the at least one static tube and the at least one static duct and the at least one first canal being aligned to form another conduit.

2. The improved catheter of claim 1 wherein the first sidewall and the second sidewall are detachably connected.

3. The improved catheter of claim 1 wherein the at least one static tube has a longitudinal axis perpendicular to the main tube's longitudinal axis.

4. The improved catheter of claim 1 wherein the casing is made of a resilient material.

5. The improved catheter of claim 1 further comprising:
a fastener;
the fastener being incident to the first sidewall and the second sidewall.

6. The improved catheter of claim 1 further comprising:
at least one suture ring;
the at least one suture ring being integrally attached to the casing.

7. An improved catheter comprising:
the catheter having a main tube;
the catheter having a casing;
the main tube having a first main tube end;
the first main tube end being integrally attached to the casing;
the main tube having at least one first canal;
the main tube having at least one second canal;
the casing having a moveable portion;
the casing having a static portion;
the casing being made from a resilient material;
the moveable portion having a first sidewall;
the static portion having a second sidewall;
the first sidewall and the second sidewall being detachably connected;
at least one moveable duct;
at least one static duct;
the at least one moveable duct being enclosed in the moveable portion;
the at least one static duct being enclosed in the static portion;
at least one static tube;
the at least one static tube having a longitudinal axis perpendicular to the main tube's longitudinal axis;
at least one moveable tube;
the at least one static tube being integrally attached to the at least on static duct;
the at least one moveable tube being integrally attached to the at least one moveable duct;
the at least one moveable tube and the at least one moveable duct and the at least one second canal being aligned to form a conduit;
the at least one static tube and the at least one static duct and the at least one first canal being aligned to form another conduit;
at least one suture ring;
the at least one suture ring being integrally attached to the casing.

8. The improved catheter of claim 7 further comprising:
a fastener;
the fastener being incident to the first sidewall and the second sidewall.
